(19) United States
(12) Patent Application Publication
(10) Pub. No.: US 2016/0379173 A1
(43) Pub. Date: Dec. 29, 2016

(54) APPOINTMENT SCHEDULING SYSTEM AND METHODS

(71) Applicant: Cardinal Health Commercial Technologies, LLC., Dublin, OH (US)

(72) Inventors: KIRAN KUMAR KARNATI, POWELL, OH (US); BRENT EUGENE STUTZ, DUBLIN, OH (US)

(73) Assignee: Cardinal Health Technologies, LLC., Dublin, OH (US)

(21) Appl. No.: 15/192,169

(22) Filed: Jun. 24, 2016

Related U.S. Application Data
(60) Provisional application No. 62/184,604, filed on Jun. 25, 2015.

(51) Int. Cl. G06Q 10/10; G06Q 30/02; G06F 17/30

(52) U.S. Cl. CPC G06Q 10/1095; G06F 17/3053; G06Q 30/0235

(57) ABSTRACT

Methods and systems are described for providing a marketplace between consumers (e.g., patients, parents, guardians, pet owners, and/or other users) and providers of services, such as health services. The marketplace method and system may identify appointment availability, provide ratings for service providers, provide prices for the services delivered, identify and indicate locations for providers, facilitate scheduling of appointments, and provide a payment handling process for the services provided. In addition, the marketplace may provide bi-directional communications between consumers and providers.

### PRIORITY

This application claims priority to U.S. Provisional Patent Application No. 62/184,604 filed on Jun. 25, 2015, titled “APPOINTMENT SCHEDULING SYSTEM AND METHODS.”

### FIELD

Aspects of the present disclosure relate to scheduling appointments and related activities. In particular, for example, aspects of the disclosure relate to a system for scheduling appointments and setting pricing/payment, among other features, with service providers, such as doctors, dentists, veterinarians, and other professionals.

### BACKGROUND

Generally, people schedule appointments for various services by calling the service provider and inquiring about available time slots. For example, a patient may call a dentist to schedule an appointment for a cleaning and may need to schedule a subsequent appointment for another procedure such as a filling or root canal. Similar appointment reservation systems are used for physicians, veterinarians, optometrists, cosmetic surgeons, home health services, outpatient surgeries, and other service providers.

Such telephone scheduling may limit a consumer's choices regarding service providers because of the burden and other limitations of calling multiple service providers. Moreover, pricing information may not be available.

From the service provider's perspective, current scheduling systems may leave gaps in the service provider's schedule where the service provider earns no revenue. General promotions or discounts may increase the number of appointments and fill the gaps, but may also reduce revenue from other appointments.

In view of the foregoing, improvements to scheduling systems for service providers may be desirable.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its purpose is to present some concepts of one or more aspects in a simplified form as a prelude to the more detailed description that is presented later.

The described aspects relate to methods and systems for providing a marketplace between consumers (e.g., patients, parents, guardians, pet owners, and/or other users) and providers of health services. Health services may include, but are not limited to, dental services, vision services, primary care services, surgeries, outpatient procedures, alternative medicine services, home health services, telemedicine services, nursing home services, cosmetic procedures, and veterinary services. The marketplace method and system may identify appointment availability, provide ratings for service providers, provide prices for the services delivered, identify and indicate locations of providers, facilitate scheduling of appointments, and provide a payment handling process for the services provided. In addition, the marketplace may provide bi-directional communications between consumers and providers.

The described aspects may provide a software platform for consumers and providers to access the marketplace. The platform may be accessible through a mobile application and/or a web application. For example, a consumer may access the platform via a mobile application, and a provider may access the platform through the web application.

Portions of an example method and system are provided below.

Additional advantages and novel features relating to aspects of the present invention will be set forth in part in the description that follows, and in part will become more apparent to those skilled in the art upon examination of the following or upon learning by practice thereof.

### BRIEF DESCRIPTION OF THE DRAWINGS

The disclosed aspects will hereinafter be described in conjunction with the appended drawings, provided to illustrate example features and not to limit the disclosed aspects, wherein like designations denote like elements, and in which:

FIG. 1 illustrates an example system for providing a service appointment marketplace and scheduling system in accordance with aspects of the present disclosure.

FIG. 2 illustrates an example provider interface for scheduling appointments in accordance with aspects of the present disclosure.

FIG. 3 illustrates an example provider interface for scheduling service blocks in accordance with aspects of the present disclosure.

FIG. 4 illustrates another example provider interface for scheduling service blocks in accordance with aspects of the present disclosure.

FIG. 5 illustrates an example provider interface for indicating services and prices in accordance with aspects of the present disclosure.

FIG. 6 illustrates an example provider interface for configuring discounts in accordance with aspects of the present disclosure.

FIG. 7 illustrates another example provider interface for configuring discounts in accordance with aspects of the present disclosure.

FIG. 8 illustrates a consumer interface for displaying a consumer participation information in accordance with aspects of the present disclosure.

FIG. 9 illustrates an example provider interface for displaying a practice summary in accordance with aspects of the present disclosure.

FIG. 10 illustrates a consumer interface for selecting a service in accordance with aspects of the present disclosure.

FIG. 11 illustrates a consumer interface for selecting search filters in accordance with aspects of the present disclosure.

FIG. 12 illustrates a consumer interface for displaying appointment search results in accordance with aspects of the present disclosure.

FIG. 13 illustrates another consumer interface for displaying appointment search results in accordance with aspects of the present disclosure.

FIG. 14 illustrates a consumer interface for displaying a detailed provider schedule in accordance with aspects of the present disclosure.

FIG. 15 illustrates a flowchart showing an example method of ranking appointments in accordance with aspects of the present disclosure.

FIG. 16 illustrates a consumer interface for submitting a booking request in accordance with aspects of the present disclosure.

FIG. 17 illustrates a provider interface for confirming a booking request in accordance with aspects of the present disclosure.

FIG. 18 illustrates a consumer interface for displaying pending appointments in accordance with aspects of the present disclosure.

FIG. 19 illustrates a consumer interface for displaying favorite providers and visits in accordance with aspects of the present disclosure.

FIG. 20 illustrates a consumer interface for rating a provider in accordance with aspects of the present disclosure.

FIG. 21 illustrates a provider interface for displaying consumer bids for appointments in accordance with aspects of the present disclosure.

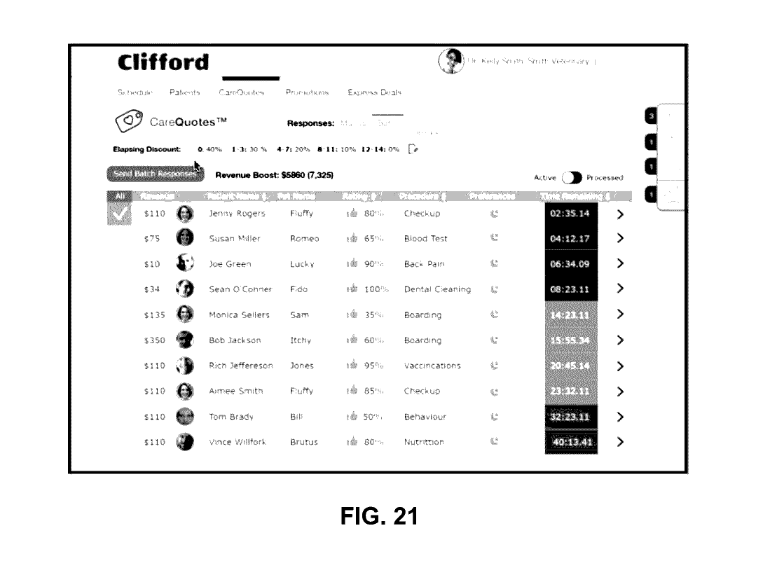

FIG. 22 contains a representative block diagram illustrating various aspects of an example computer system in accordance with aspects of the present disclosure in accordance with aspects of the present disclosure.

### DETAILED DESCRIPTION

Various aspects are now described with reference to the drawings. In the following description, for purposes of explanation, numerous specific example details are set forth in order to provide a thorough understanding of one or more aspects. It may be evident, however, that such aspects may be practiced without these specific details.

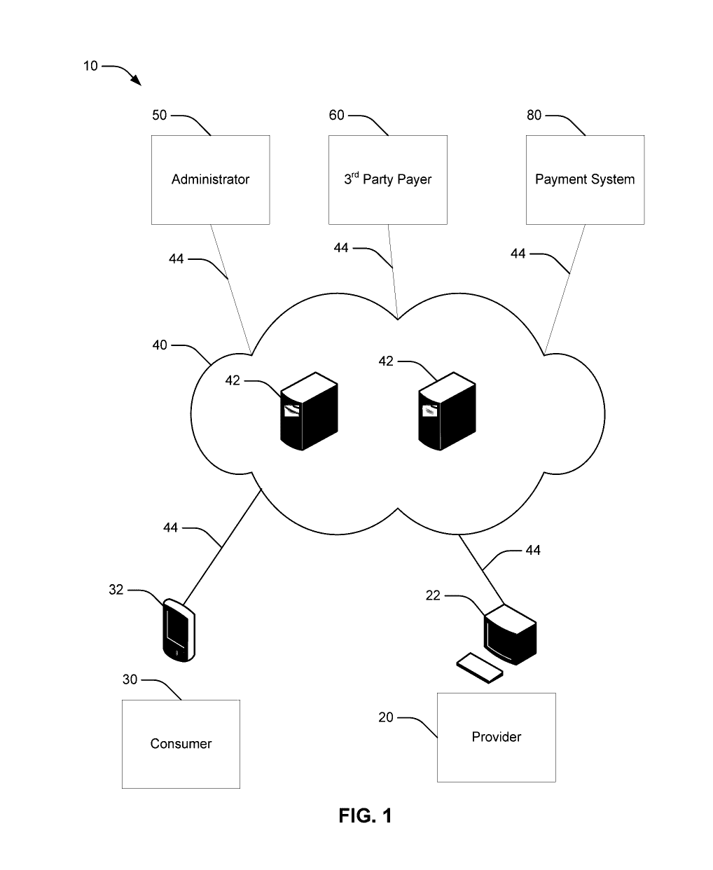

FIG. 1 illustrates an example system 10 (e.g., a software platform) for providing a service appointment marketplace and scheduling features. The system 10 may identify and present available appointments for various health services. The system 10 may allow a provider 20 to identify available appointments for specific services provided by the provider 20 through use of a calendaring type feature. The system 10 may include an interface for a provider 20 on a provider computer system 22 (e.g., a service provider module), an interface for a consumer 30 on a consumer device 32 such as a mobile phone, tablet, or computer (e.g., a consumer module), and a network 40. In an aspect, the system 10 may provide the service appointment marketplace and scheduling system via a web application accessible to multiple users. For example, the service appointment marketplace and scheduling system may be hosted on one or more computer servers 42. The network 40 may also provide access to the web application for other entities, such as an administrator 50 or third party payer 60, and/or provide an interface with a payment system 80.

In one aspect of the present disclosure, various data may be, for example, input and/or accessed by the provider 20, consumer 30, administrator 50, third party payer 60, payment system 80, or other users via terminals, such as personal computers (PCs), minicomputers, mainframe computers, microcomputers, telephonic devices, or wireless devices, personal digital assistants (“PDAs”) or a hand-held wireless devices (e.g., wireless telephones). The terminals may be coupled to a computer server 42, such as a PC, minicomputer, mainframe computer, microcomputer, or other device having a processor and a repository for data and/or connection to a repository for data, via, for example, a network 40, such as the Internet or an intranet, and/or a wireless network. The couplings 44 may include, for example, wired, wireless, or fiber optic links.

The provider 20 may provide one or more services, e.g., health services. The provider computer system 22 may be any computer system that enables the provider 20 to access a network based platform, e.g., a web service. For example, the provider computer system 22 may include one or more personal computers (PCs), minicomputers, mainframe computers, microcomputers, telephonic devices, or wireless devices, personal digital assistants (“PDAs”) or a hand-held wireless devices (e.g., mobile telephones). In an aspect, the provider computer system 22 may access the system 10 via a web browser. In an aspect, the provider computer system 22 may also include a practice management system that may, for example, perform scheduling and billing tasks for the provider 20. In an aspect, the system 10 may be operated in parallel with the provider-based practice management system. The system 10 may also be integrated with the provider based practice management system, for example, to avoid duplicate entries for appointments. For example, the system 10 may receive data (e.g., appointments) from the provider based practice management system and generate appointments within the system 10. Conversely, the system 10 may export data to various provider based practice management systems.

The provider computer system 22 (service provider module) may be communicatively coupled to a network 40. For example, the provider computer system 22 may include a modem and be coupled to the network 40 via a wired or wireless connection. The network 40 may include one or more computer servers 42. The computer servers 42 may include, for example, a database or server storing provider information, patient information, and appointment information. The provider computer system 22 may periodically update the computer server with any changes to provider information or appointment information. The provider computer system 22 may also update information based on events occurring at the provider computer system 22 (e.g., patient check-in or completed appointments).

The consumer 30 may be anyone seeking a service provided by one or more providers 20. In an aspect, the consumer 30 may be a patient seeking a medical service. The consumer 30, however, may also include a representative of a patient, such as a spouse, a caregiver, a parent or guardian of a child, or a pet owner. The system 10 may include accounts for consumers, which may include one or more sub-accounts for patients a particular consumer 30 is responsible for.

The consumer device 32 may be any computer system that enables the consumer 30 to access a network based platform, e.g., a web service. In an aspect, the consumer device 32 may be capable of executing an application specifically for providing an interface to the network based platform. For example, the consumer device 32 may be a wireless device (e.g. smart phone or tablet) running an installed application. In another aspect, the consumer device 32 may use a web browser to present a similar web-based interface. In an aspect, the consumer device 32 may include a location component capable of determining a current, real-time location of the consumer device 32. For example, the consumer device 32 may include a global positioning system (GPS) receiver or a cellular telephone locating feature. The consumer device 32 may also use a network address or consumer entered location to determine the current location.

In an aspect, the consumer device 32 may be used to control access to patient medical records. The patient medical records may be stored on the consumer device 32, a server 42, and/or one or more service provider computer systems 22. A consumer 30 may be able to request and access a medical record stored anywhere within the system 10 using the consumer device 32. The consumer 30 may also control whether another entity may access a medical record. For example, a request from a service provider 20 to view a medical record may require approval from the consumer 30 via the consumer device 32. Accordingly, a consumer 30 may, for example, release a medical record to a new service provider before a scheduled appointment.

The administrator 50 may be capable of accessing information corresponding to other users. For example, the administrator 50 may be able to access account information of a provider 20 or consumer 30 in order to provide support. The role of the administrator 50 may be limited because the providers 20 and consumers 30 may drive the marketplace. For example, the administrator 50 may be able to view information, but may be unable to autonomously change provider or consumer information. As another example, the administrator 50 may be prevented from viewing certain information, such as patient medical records.

The third party payer 60 may be any party responsible for paying for all or part of services provided by a provider 20 to a consumer 30. For example, a third party payer 60 may include an insurance provider or government agency. The third party payer 60 may receive billing information from the network 40 and provide payment and/or partial payment. For example, a server 42 may be a billing server that receives bills from the provider 20 and provides the bills to the third party payer 60. In an aspect, the third party payer 60 may provide a schedule of payments for various services. When determining prices corresponding to available appointments, the system 10 may apply the third party payer schedule of payments to provider-specified prices to determine prices to be paid by the consumer 30.

The payment system 80 may include any financial services network. The payment system 80 may allow consumers 30 to pay for services. In an aspect, the payment system 80 may first process a payment from a consumer 30 to the system 10. For example, the consumer 30 may make a payment via the system 10 upon completion of an appointment during a check-out process. The payment system 80 may also process a payment from the system 10 to a provider 20. The system 10 and/or the payment system 80 may deduct a fee when paying the provider 20. Payments may be made using different payment types. For example, a payment from a consumer 30 via the system 10 may include use of a credit card, debit card, digital currency, online account (e.g., PayPal or Bitcoin) or other payment service or feature. A payment via the system 10 to a provider 20 may use a direct transfer, electronic check, automated clearing house (ACH), or other payment service or feature. Multiple payments to a provider 20 may optionally be pooled into a single transaction. In an aspect, the payment system 80 may include a payment gateway that provides access to multiple financial service networks. For example, the payment system 80 may be configured to store payment information for consumer 30 and process payments using the stored payment information. The payment system 80 or payment gateway may provide the system 10 with a token to identify the payment information of the consumer 30. In another aspect, the payment system 80 may allow payments by a consumer 30 as a guest. A guest may not need to register an account with the system 10. Instead, for example, a guest may book an appointment with a provider 20 through the system 10 and pay via a credit card. The guest may optionally provide contact information associated with a single appointment.

### Available Appointments

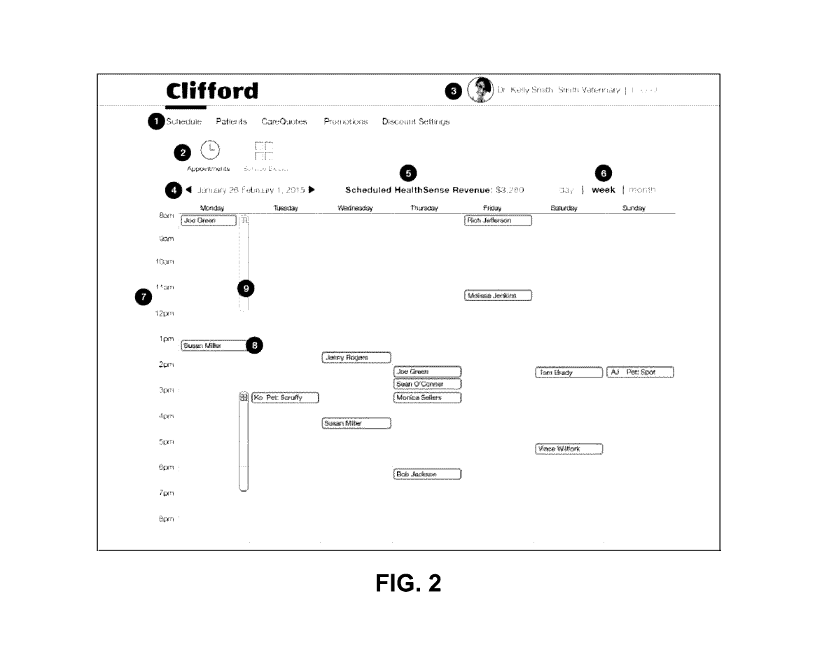

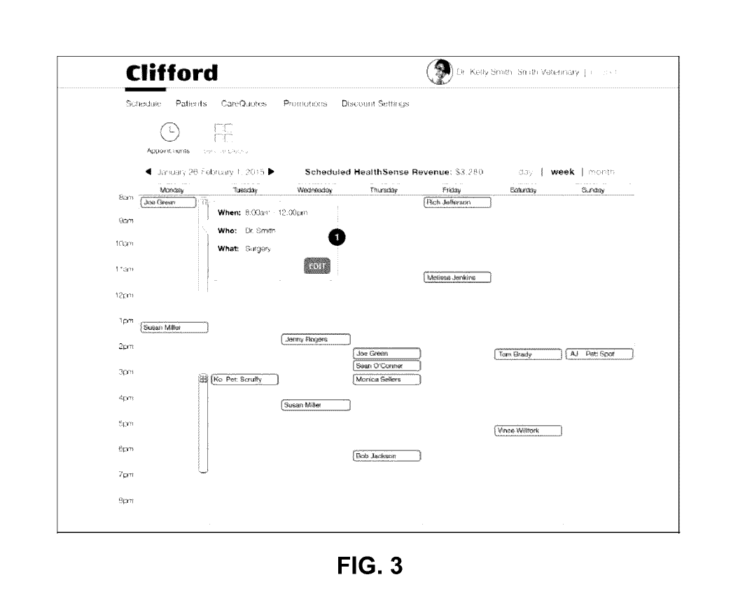

As illustrated in FIG. 2, the provider 20 may use a calendar, for example, a calendar stored on the server 42 and displayed on the provider computer system 22 via a web application, to identify presently available appointments. For example, existing appointments may be blocked out in the calendar and display the name of a consumer 30 scheduled for the appointment time and the type of procedure to be performed. Available appointments may appear as blank spaces on the calendar. The calendar may selectively display a day, week, month, or a custom time frame. In addition, the provider 20 may use the system 10 to identify available service blocks for a specific service. For example, as illustrated in FIG. 3, the service block may identify the type of service (e.g., surgery) and the available time range for the service. The system 10 may allow only appointments for the identified type of service to be scheduled during the service block. The provider 20 may adjust or move the service block within the calendar to designate the desired time. The service block may also be edited manually to change or add service types. The calendar may present a day, week, month, or custom view of the provider's appointments. In addition, the calendar may present certain financial information, such as a sum of charges for the appointments currently booked through the system 10 for a displayed time frame, a percentage of total time slots that are booked, etc.

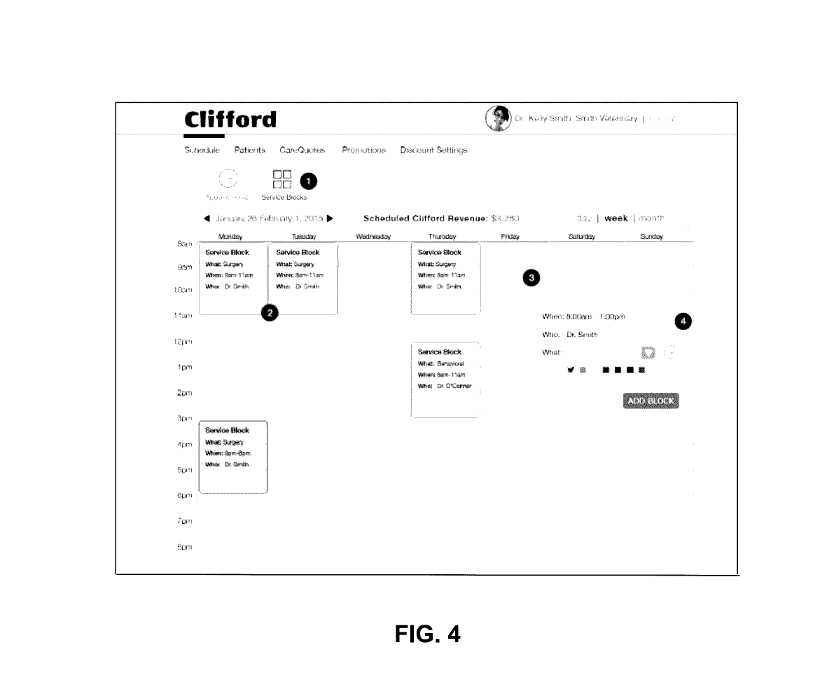

The provider 20 may, for example, drag and drop appointments and/or service blocks into the system 10 so that the system 10 may determine the appointment availability for the provider 20. As illustrated in FIG. 4, the provider 20 may also select a time to enter a new appointment and/or service block into the system 10. The system 10 may also integrate with a provider's calendar (e.g., a calendar used in a provider practice management system) to identify the available appointments and/or service blocks. In addition, the system 10 may allow a provider 20 to limit the appointments that public users (e.g., consumers 30) may view. For example, the provider 20 may only allow the consumers 30 to view appointments in the afternoon. The changes the provider 20 makes to the calendar may automatically and instantly populate in the marketplace as available and/or unavailable appointments. A consumer 30 may use a mobile application on the consumer device 32 to view available appointments for various providers 20 and selectively schedule an appointment.

### Prices for Services

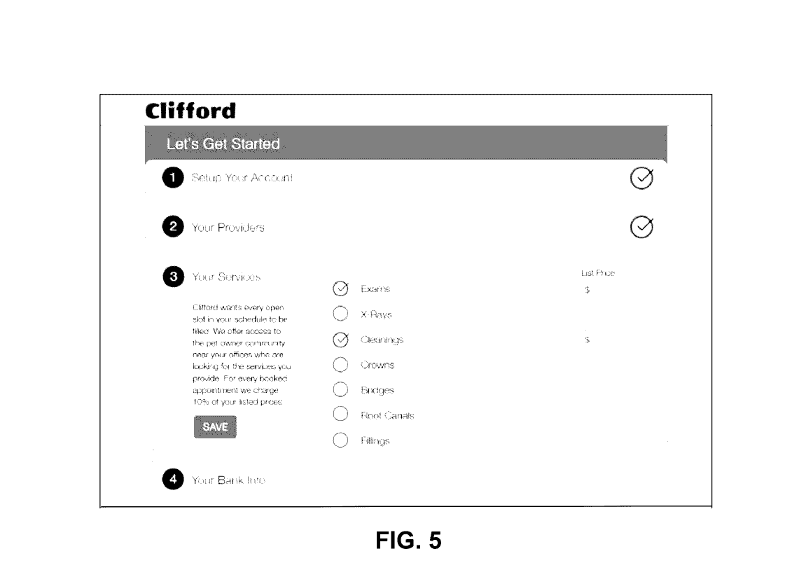

In addition to scheduling appointments, the system 10 may allow a provider 20 to identify prices for the services provided. The provider 20 may access the system 10 to define various prices for the services that the provider 20 offers. For example, the system 10 may identify the provider 20 by service type and category. As illustrated in FIG. 5, for example, the system 10 may then present a list of potential services that may be offered through the system 10. The provider 20 may then enter a base list price for each service offered by the provider 20. Different providers 20 of the same service may offer different prices for the services provided (including different providers within a single practice).

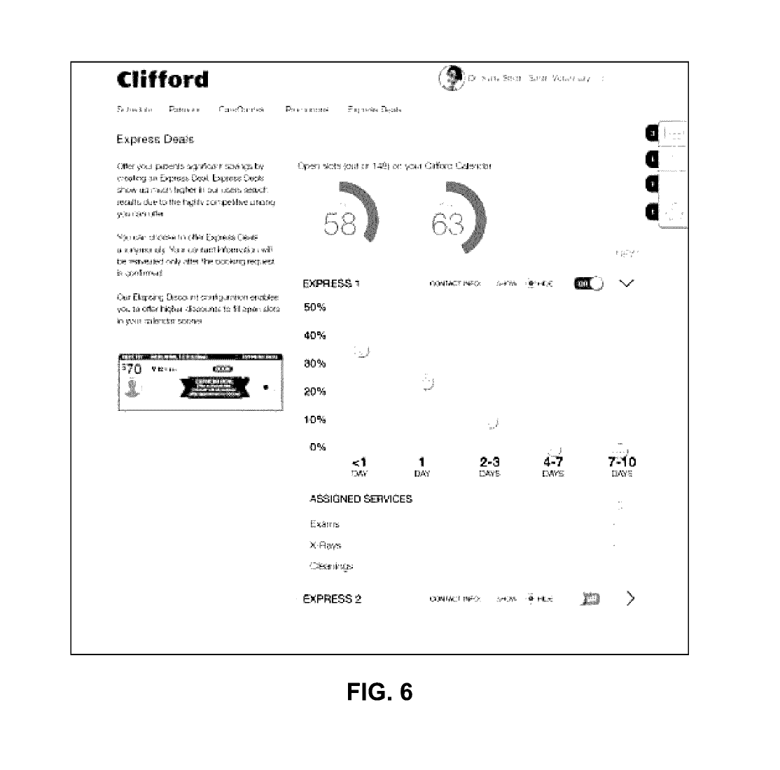

The system 10 may also allow a provider 20 to selectively discount the price for the services provided. For example, as illustrated in FIG. 6, a provider 20 may configure an express discount to target upcoming open appointment times. The provider 20 may offer a large discount for upcoming same-day appointments that would otherwise go unfilled. The provider 20 may offer lesser discounts for next-day and same-week appointments. It should be appreciated that the provider 20 may configure discounts based on the particular nature of the practice and current business needs. As another example, the discount may be based on the time of day. For example, if a provider 20 typically has a time of day that is slow (e.g., from 1 pm to 2 pm), the provider 20 may offer a higher discount on services provided during the slow time than prices offered on services during a peak time of day. In addition, if a provider 20 has a last minute cancelation, the provider 20 may offer a higher discount on the services to fill the last minute cancelation.

The discount may be configured for particular services. The various services offered by a provider 20 may have different overhead or marginal costs, and a provider 20 may vary the discount offered for each service based on the relevant costs. For example, a dentist may offer up to a 25% discount on an examination, while the dentist may only offer up to a 15% discount on an x-ray procedure because of higher overhead or marginal costs for the x-ray procedure. The discount may also vary based on the demand for the time slots or services. For example, a more popular service may have a lower discount than a less popular service. As such, the provider may be able to drive traffic to specific service blocks by offering discounts on specific services.

In addition, the discounts provided may be configured as “lapsing discounts.” For example, the discount may only apply for a specific time on a specific day. As such, the system 10 may periodically recalculate prices based on lapsing discounts. In addition, discounts may change during the day based on consumer demand. For example, the discounts may increase and/or decrease based on consumer demand for a particular day and/or service. In an aspect, the system 10 may calculate the consumer demand for a particular service for a time period based on the number of available appointments during the time period for the provider 20 and/or other providers offering the same service. Accordingly, a provider 20 may offer a relatively large discount when there are a large number of appointments to fill and gradually reduce the discount as the provider's schedule fills up.

The parameters for the discounts may be preconfigured into the system 10. For example, a provider 20 may identify parameters for the discount offered for each service. Parameters may include, for example, a range for the discount (e.g., 10 to 25%), weights for different time slots (e.g., afternoon appointments may receive a higher discount than morning appointments), and weights for last minute cancelations (e.g., a higher discount may be applied to a last minute cancelation resulting in an open appointment). For example, if a provider 20 has a last minute cancelation in a low demand time period, the system 10 may apply the highest discount the provider 20 is willing to give to the service and notify the consumers 30 of the available appointment and the price for the service. However, if the provider 20 has an open time slot during a high demand time, the system 10 may apply a lower discount to the service.

In an aspect, the discounts may be based on or correlated with a rating of a provider 20 and/or a rating of a consumer 30. For example, the system 10 may default a provider 20 with a relatively low ranking to offer a relatively large discount to attract customers. Conversely, the system 10 may default a relatively high ranking provider 20 to offer a smaller discount. The system 10 may, for example, suggest a discount for a particular provider 20, in order to be competitive with other providers in a geographical area. For example, the system 10 may suggest a discount that would result in the provider moving up in search result rankings, as discussed in further detail below. The provider 20 may determine whether to use the suggested discount. For example, the provider 20 may manually adjust a discount suggested by the system 10.

In another aspect, discounts may be offered based on consumer ratings. For example, a provider 20 may indicate that a discount (or an amount of a discount) is only available to consumers 30 that have a minimum threshold rating. As another example, a provider 20 may configure a discount to scale based on the consumer's rating. For example, a discount percentage may be multiplied by the rating of the consumer 30. Accordingly, the provider 20 may configure discounts based on consumer ratings to attract desirable consumers 30 and/or avoid problematic consumers 30.

The system 10 may allow the provider 20 to offer a discount anonymously. For example, a provider 20 may wish to fill appointment slots by offering a large discount without damaging the provider's reputation or letting potential consumers 30 know that large discounts are available by waiting until the last minute. The contact information of the provider 20 may be revealed only after the booking request is confirmed. Accordingly, only consumers 30 who actually book the service may be made aware that the specific provider 20 offered the discount.

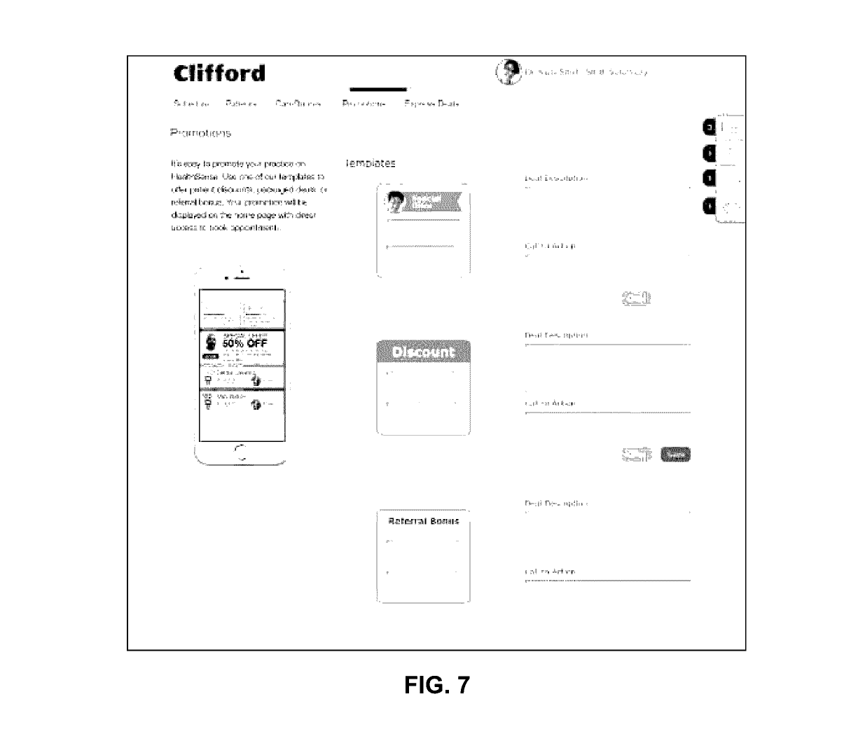

As illustrated in FIG. 7, the system 10 may provide templates that the provider 20 may use when defining parameters for the various discounts and/or promotions. For example, templates may be provided to offer promotions such as special offers, discounts, bundled services for the entire treatment cycle of a condition, and referral bonuses. The templates may ask for required parameters for the promotion. The system 10 may generate promotions for viewing by users based on the completed templates.

In an aspect, the system 10 may offer a discount, rewards, or credits instead of or in addition to a discount offered by a provider 20. For example, the system 10 may offer a discount based on repeated use of the system or repeated appointments with a provider 20. Alternatively, the system 10 may offer a discount after completing a requisite number of appointments while attaining a requisite average rating. For example, the system 10 may award credits and/or another form of rewards points to consumers 30 and providers 20 who use the system 10 frequently (e.g., book appointments, provide services, rate providers, or share experiences). The credits may be redeemed for discounts on future services, prizes, and/or other rewards. The system 10 may also establish goals for the consumer 30 or provider 20 to accomplish (e.g., booking a number of appointments, booking a same day appointment). The consumer 30 or provider 20 may be awarded credits or other rewards when a goal is accomplished. The system 10 may also track a consumer's use of the system through credits, levels, and/or badges, for example.

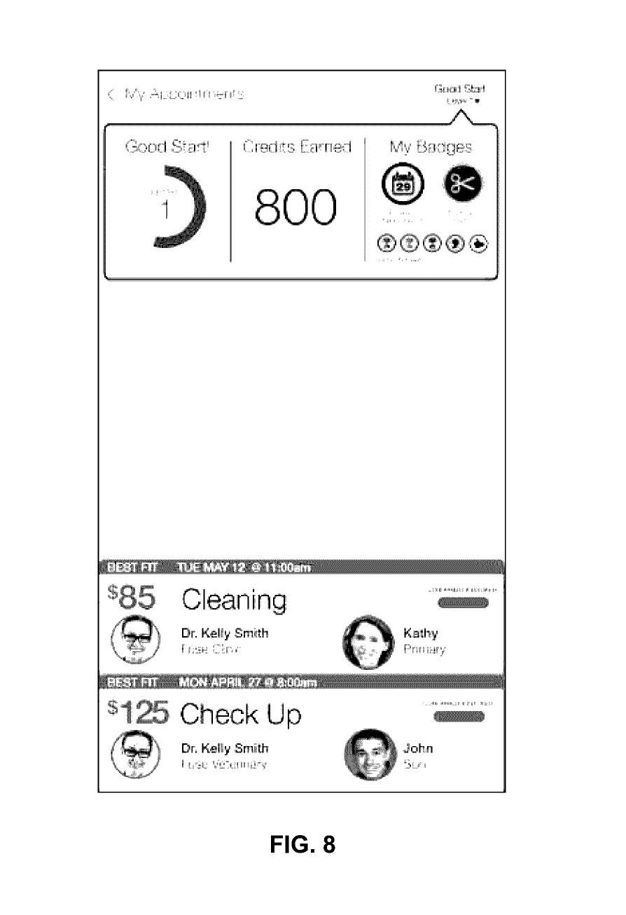

FIG. 8 illustrates an example consumer interface for displaying a consumer's participation showing a level, credits, and badges of the consumer 30. The interface may be accessed from another consumer interface, for example, via a participation icon displayed on the other interface. A consumer 30 may earn credits for some or every action performed using the system 10. A consumer's level may be based on a total number of credits the consumer has earned. The system 10 may award badges to consumers 30 for accomplishing specific tasks or goals. The system 10 may use a similar methodology to track the participation of providers 20. The system 10 may offer discounts, for example, based on the consumer's credits, level, or badges. For example, the system 10 may offer a discount on the next appointment scheduled after earning a badge.

The system 10 may automatically or selectively apply the various rules to the available appointments for the provider 20 and calculate the discounts or rewards for the appointments available that day. As such, when a consumer 30 searches for available appointments, for example, the consumer 30 may view the available appointments and the price (with the discount or rewards included) for the services requested. In an aspect, the system 10 may calculate the price for each appointment based on the applicable discounts when the consumer 30 searches for the service.

### Practice Summary

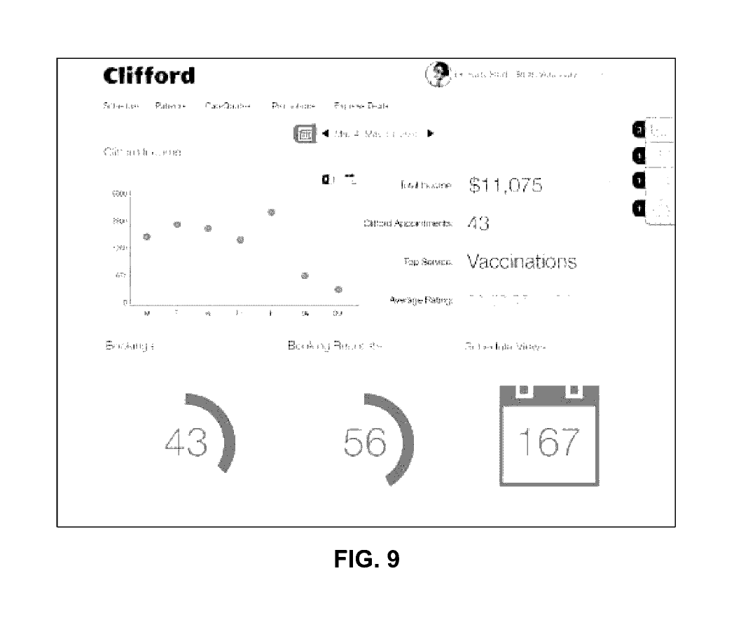

The system 10 may provide practice summary information for use by providers 20. The practice summary information may include consumer activity analytics and/or financial analysis. For example, as illustrated in FIG. 9, the practice summary may include the total income generated from appointments booked through the system 10, a list of patients, a number of booking requests received, a number of appointments booked through the system 10, a number of times the provider's page/schedule was viewed by users, rankings of services booked through the system 10, and the provider's average rating (e.g., inputted ratings by consumers 30, as described further below). The practice summary may also include analysis of revenue, discounts, and fees charged by system 10. The practice summary information may be exportable to various devices or file formats for analysis.

### Searching for Appointments

A consumer 30 may use the system 10 to search for available appointments for a service within a certain distance from the consumer 30. For example, the system 10 may provide a series of menus to sort between service types, categories, and specific services. For example, service types may include: medical, dental, veterinary, chiropractic, cosmetic, nutrition, and/or fitness. Categories may correspond to various specializations within a service type. For example, general dental services, oral surgery, and orthodontic services may be different categories within a dental service type.

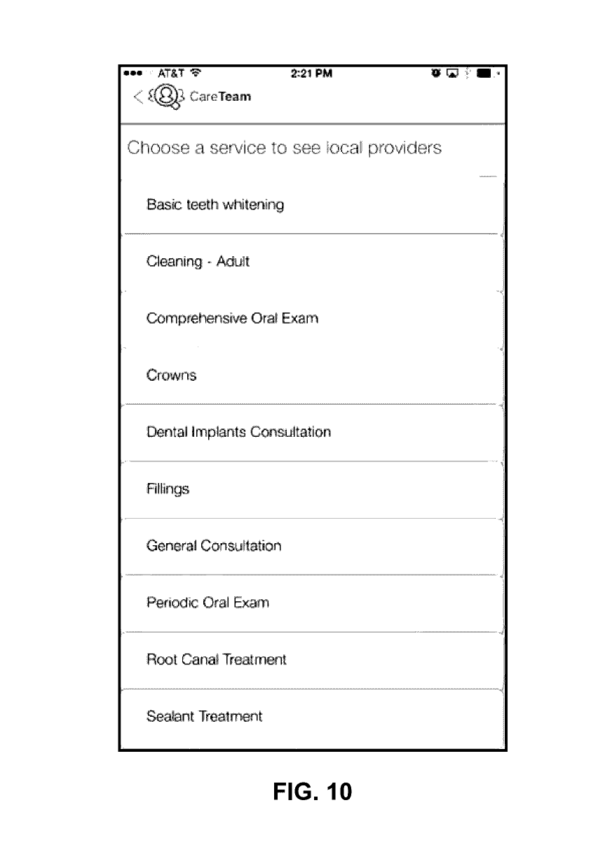

FIG. 10 illustrates an example of a menu of services that may be provided within a general dental services category. In an aspect, a service may be unique to a service type and/or category. For example, a periodic oral examination provided within a dentistry service type and in a general dental services category may be different from a periodic oral examination provided within a veterinary service type and in a canine category. A provider 20 may register to provide services in different service types and/or categories.

The system 10 may automatically detect the location of a consumer 30, for example, using information gathered from a GPS-component or other location component on the consumer's wireless device. In addition, the consumer 30 may enter in a zip code or other location information into the system 10 to provide the consumer's location. The consumer 30 may use a default distance (e.g. 15 miles) or change the distance parameter for the search.

In an aspect, the consumer 30 may also refine and/or filter the search by entering additional search criteria. For example, as illustrated in FIG. 10, the system 10 may present options for selecting additional search criteria, such as an indication of whether the provider 20 is accepting new patients, ratings above a certain level (e.g., above 3 stars), appointments within a certain time frame (e.g., preferred days of the week, and/or times of day), price (e.g., below a certain price), and distance (e.g., increase and/or decrease the maximum distance from the user's location). In an aspect, the additional search criteria may be applied by a server 42 (FIG. 1) such that only search results meeting the filter are shown to the consumer 30. In another aspect, the consumer device 32 (FIG. 1) or web application may receive a complete set of search results and apply the additional search criteria to the received search results, for example, after the consumer 30 has seen at least a requisite number of search results. As another example, the system 10 may allow a consumer 30 to pre-rank their preferences based on various ranking parameters (e.g., cost, distance, rating, and time).

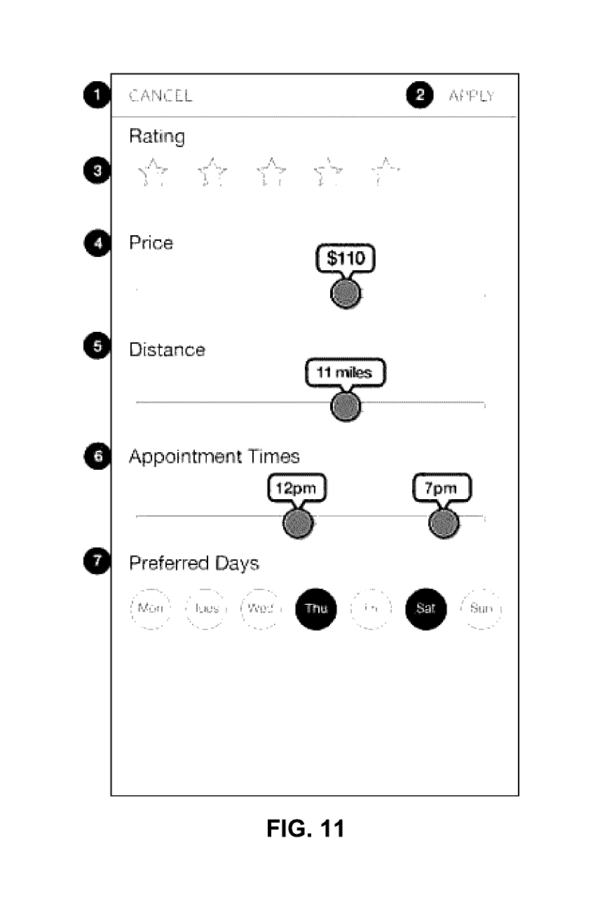

The system 10 may present a plurality of available appointments based on the search requested by the consumer 30. For example, as illustrated in FIG. 11, the system 10 may present the name of the selected service at the top of the search results list, along with an average cost for the appointments presented. The system 10 may automatically rank the plurality of available appointments and present the highest ranked appointments to the consumer 30 (see additional information on ranking, below). The consumer 30 may request additional search results, for example, by swiping top to bottom on the display screen (or otherwise making a selection) to produce additional search results or selecting an icon to display additional search results.

The appointments presented may include a summary of the provider 20 and the service. For example, the summary may include an appointment price, the provider's name, picture and address, distance (e.g., determined by comparing the consumer's location relative to the identified provider's location), ratings of the provider 20, a map displaying the current location of the consumer 30, as well as each provider's location, and a “Book” button that the consumer 30 may select for booking the available appointment.

The system 10 may present the available appointments in a computed order and/or rank the available appointments. For example, the available appointments may be ranked by the system 10 based on weighting of one or more parameters, such as the lowest price, quality of the provider 20, distance from the consumer/selected location, and/or soonest date/time available. The system 10 may automatically classify the available appointments into groups. For example, the system 10 may identify the best available appointments by highlighting the appointments in green, the next best appointments by highlighting the appointments in orange, and less desirable appointments by highlighting those appointments in red (or other suitable colors).

The system 10 may automatically or otherwise vary the weightings based on the consumer's selection history. For example, if the consumer 30 exhibits a pattern of selecting higher rated service providers 20 as compared to nearby providers, the system 10 may classify the consumer 30 as a “quality-sensitive” consumer, increase the weighting preference factor for rating, and decrease the weighting preference factor for distance. As another example, if the consumer 30 exhibits a pattern of selecting the geographically nearest service provider, the system 10 may classify a consumer 30 as a “convenience-sensitive” user and increase a weighting preference factor for closest distance. As yet another example, if the consumer 30 exhibits a pattern of selecting a lowest-price provider 20, the system 10 may classify the consumer 30 as a “price-sensitive” user and increase a weighting preference factor for lower price.

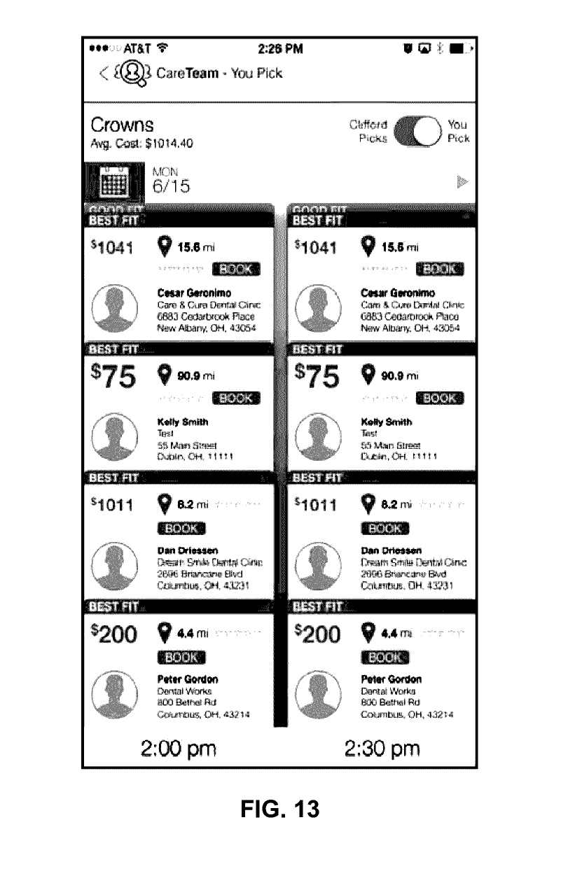

In another aspect, the system 10 may allow the consumer 30 to choose a specific time or date. For example, as illustrated in FIG. 13, the consumer 30 may select a “You Pick” mode that displays available appointments for a specific date and times. The appointments may be ranked for each time slot based on various parameters, such as price, distance, and provider quality/rating. The system 10 may display multiple appointments for a provider 20 if appointments are available in different time slots. The consumer 30 may view later/earlier times by swiping the screen right to left, or left to right, for example, and/or selecting an icon displayed with the search results to change the times. In addition, the consumer 30 may view different dates by selecting a calendar icon and selecting a different date. The default date for the result list may be the current date. The appointments in the “You Pick” mode may also include an appointment summary. In an aspect, the appointment summary may be condensed to allow more appointments to fit on a display.

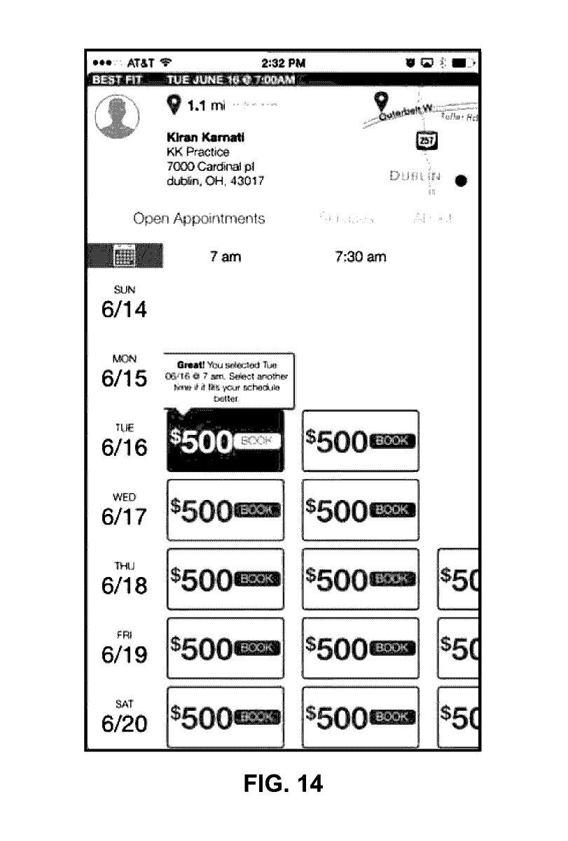

Selecting the book button may present detailed provider information for the selected provider 20. For example, if a consumer 30 wants to view additional appointments available for a presented provider 20, the system 10 may present additional provider information in a separate page. FIG. 14 illustrates an example display of detailed provider information. Additional provider information may include, for example, the provider details from the summary page, open appointments, all or a range of services provided by the provider 20, and information about the provider 20. The open appointments may include a calendar view with the available appointments for the provider 20. The system 10 may present the price for the service in the available time slot and a book button next to the price, which may allow, for example, the consumer 30 to quickly and easily book the available appointment. When a consumer 30 selects an available appointment, the system 10 may change the color of the appointment to indicate that the appointment has been selected and/or provide a notification, such as a popup, text message, or e-mail to notify the consumer 30 that the appointment has been selected. The consumer 30 may select additional or different dates by entering in different dates, for example, through a displayed calendar icon. In addition, the consumer 30 may select different dates and/or times by, for example, swiping the screen right to left for different hours and/or up or down for different dates.

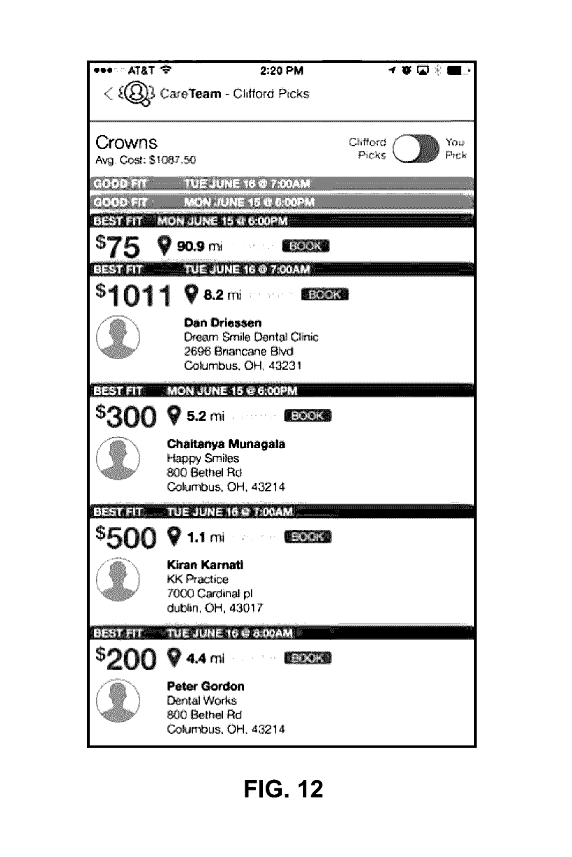

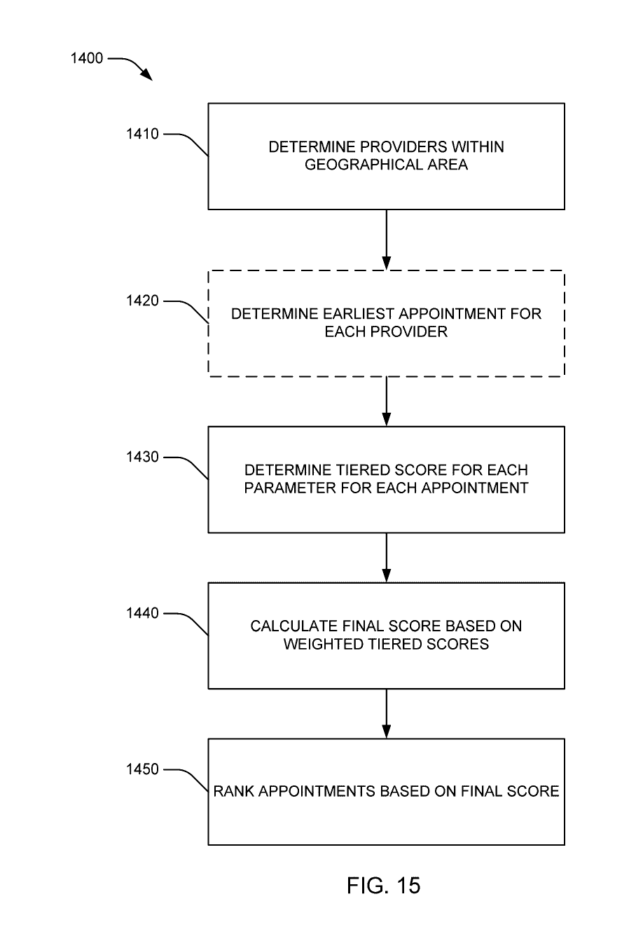

FIG. 15 is a flowchart illustrating an example method 1400 of ranking appointments shown for example in FIGS. 12-13. In an aspect, the method 1400 and related methods may be performed by a computer system 10, such as a server 42 (FIG. 1). In an aspect, the method 1400 may be performed by a consumer device 32 (FIG. 1). While, for purposes of simplicity of explanation, the methods are shown and described as a series of steps or acts, it is to be understood and appreciated that the methodologies are not limited by the order of acts, as some acts/steps may, in accordance with one or more aspects, occur in different orders and/or concurrently with other acts/steps from that shown and described herein. For example, it is to be appreciated that a methodology could alternatively be represented as a series of interrelated states or events, such as in a state diagram. Moreover, not all illustrated acts/steps may be required to implement a method in accordance with one or more aspects.

In block 1410, the method 1400 may include determining providers 20 within a geographical area. In an aspect, the server 42 (FIG. 1) may receive a selection of a service and a geographical area from a consumer device 32 (FIG. 1). The service may be a service selected by the consumer 30 from, for example, a list of services, as illustrated in FIG. 10. The geographical area may be centered at a location (e.g. GPS coordinates) of the consumer device 32 (FIG. 1). The geographical area may also include a distance from the consumer device, which may be a default value or a value selected by the consumer 30. In another aspect, the geographical area may also be indicated as a zip code, city, county, state, or other designation of a place. The server 42 (FIG. 1) may access a provider database to determine providers 20 near or within the geographical area. For example, the server 42 (FIG. 1) may compute a distance between the location of the consumer 30 and a location of the provider 20. If the computed distance is less than the selected distance value, the provider 20 may be considered within the geographical area, or ranked based on the proximity to the consumer 30 within the geographic area. In another aspect, for example, a consumer zip code may be compared to provider zip codes to select the providers 20 within the geographical area. Providers 20 in zip codes proximal or adjacent to the consumer's zip code may be regarded by the server 42 as being within the geographic area.

In block 1420, the method 1400 may optionally include determining an earliest appointment date or time for each provider 20. For example, the server 42 (FIG. 1) may acquire a single appointment having the earliest available time for each provider. In an aspect, the earliest available time may be based on the current time, or may include a minimum waiting period (e.g., 1 hour) to allow for travel. The server 42 (FIG. 1) may acquire parameters associated with the earliest available appointment for each provider 20. In an aspect, for example, the parameters may include price, quality/rating of the provider 20, distance from the consumer/selected location, and/or soonest time available.

In block 1430, the method 1400 may include determining a tiered score for each parameter for each appointment. In an aspect, for example, the server 42 (FIG. 1) may sort the appointments according to a primary parameter. For example, the server 42 may order the appointments based on price. If two appointments have the same value for the primary parameter, a secondary parameter may be used to break the tie. Alternatively, for example, the appointments may be given the same tiered score. The server 42 may group the sorted appointments by tiers. In an aspect, the tiers may be defined by percentiles. For example the top 20% may be in a top tier, the next 20% in a second tier, and so on. In another aspect, the tiers may be defined by threshold values. For example, tiers for a quality/rating parameter may be defined by a whole number of stars. Each of the appointments in a tier may receive the same tiered score for the parameter.

In block 1440, the method 1400 may include calculating a final score based on weighted tiered scores. For example, the server 42 (FIG. 1) may calculate a weighted average of the tiered score by applying weights to each of the parameters. In an aspect, the weights applied to each parameter may be fixed. In another aspect, the system 10 may dynamically or adaptively learn consumer preferences based on past selected appointments and adjust the weights based on a particular consumer's preferences or the preferences of a plurality of consumers (e.g., within a geographic area). For example, for a consumer 30 who regularly picks the lowest price appointment, the system 10 may assign a higher weight to the price parameter tiered score. As another example, if a consumer 30 consistently travels a further distance and/or pays a higher price to see a higher rated provider 20, the system 10 may apply a higher weight to the quality/rating parameter. As such, the system 10 may self-learn the consumer's preferences and adjust the available appointments presented to the consumer 30 based on the consumer's past behavior (and/or the behavior of other users in the geographic area).

In block 1450, the method 1400 may include ranking the appointments based on a final score. In an aspect, for example, the server 42 (FIG. 1) may rank the appointments in descending order according to the final score for each appointment. The server 42 may further categorize the appointments based on the final score. For example, the server 42 may categorize the appointments as “Best Fit,” “Good Fit,” or “Worst Fit.” It should be apparent that other categories may be used. In an aspect, the server 42 may determine the categories by fitting the final scores to a standard distribution or a bell curve. The server 42 may, for example, categorize the appointments based on a standard deviation from a mean score. In another aspect, for example, the server 42 may categorize appointments based on defined percentile thresholds. The server 42 may provide the ranked appointments to the consumer device 32.

The system 10 may also rank appointments for the “You Pick” mode using the method 1400. In an aspect, when the “You Pick” mode is selected, the system 10 may, as above, rank the appointments for each time slot. Accordingly, the optional block 1420 may be skipped when the “You Pick” mode is selected, and the method 1400 may be applied to each time slot. Further, when the consumer 30 selects the specific time period, the soonest date/time available parameter may be excluded from the ranking method or given no weight. Alternatively, for example, because all of the appointments within a time slot may have the same date/time available, the appointments may receive the same tiered date/time available score, so the date/time available parameter may not affect the end ranking.

### Booking an Appointment

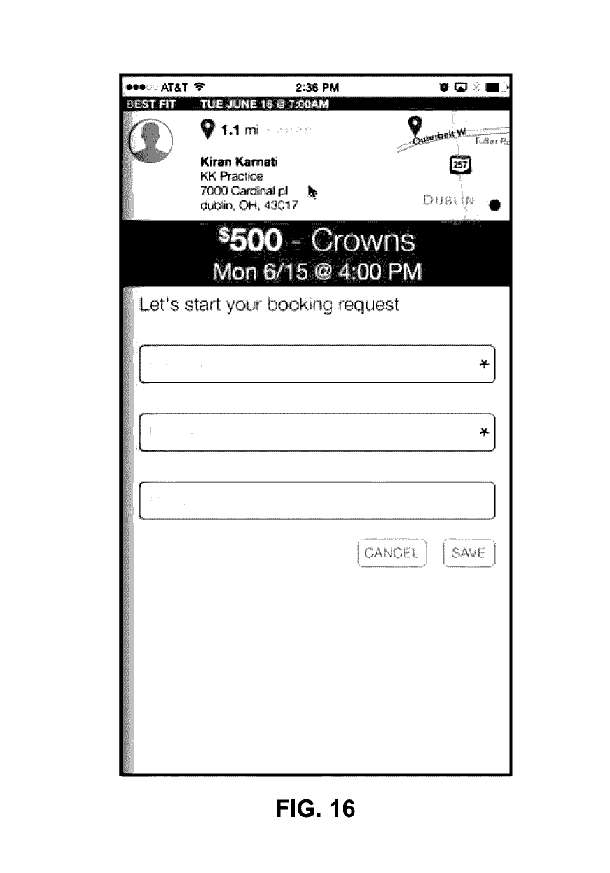

Once a consumer 30 books an appointment, for example, by selecting the “Book” button, an appointment summary may be presented to the consumer 30. FIG. 16 illustrates an example appointment summary display. The appointment summary may display the provider's information, the price for the service, and the date of the appointment, for example. The appointment summary may also request information about the patient, such as the patient name, age, and contact information (e-mail address, telephone number). This information may be used for an appointment record to be created, for example. In an aspect, the patient may be the consumer 30 or someone under the care of the consumer 30 (e.g. a child or pet). The system 10 may verify the information provided and notify the consumer 30 if changes need to be made to the information. The system 10 may also send a text message or e-mail confirmation to the consumer 30.

Once the appointment is booked, the system 10 may immediately, in real-time, show that appointment time as no longer available for other consumers to book. Alternatively, the system 10 may include a lag time (e.g., five minutes) before showing an appointment time as unavailable to allow other consumers 30 who were considering that appointment time to complete their booking. As further discussed below, a provider 20 may choose to confirm between two appointments made for the same date and time.

In addition, the system 10 may acquire payment information from the consumer 30 to store in the consumer's account, such as patient personal information, patient medical history information (e.g., provided by a questionnaire from one or more providers), credit card details, PayPal or similar payment system account information, bank details, and/or other mechanisms for payment. The system 10 may validate the information provided by the consumer 30 and notify the consumer 30 if changes need to be made to the payment information, for example.

### Appointment Confirmation

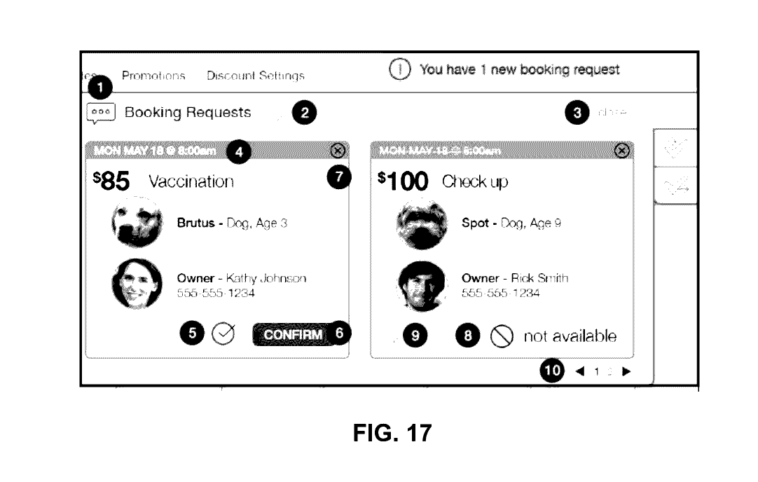

FIG. 17 illustrates an example provider interface for displaying a notification toolbar, including a booking request toolbar. The notification toolbar may also display, for example, notifications for patient check-in requests, patient check-out requests, and any other process awaiting action by the provider 20. When a consumer 30 books an appointment using the system 10, the system 10 may optionally send an appointment booking request to the provider 20 to accept the appointment. The provider 20 may either confirm and/or deny the request for the appointment. The booking request notifications may be accumulated in the notification toolbar so that the provider 20 may view the outstanding actions that the provider 20 may need to take. A booking request notification may include the time and date of the requested appointment, service to be provided, price of the appointment, and patient or consumer information, for example.

The system 10 may also check the availability of the appointment against other received booking requests and manually entered appointments (e.g., appointments scheduled by telephone and entered by the provider 20). The system 10 may display conflicting appointment requests so that a provider 20 may select which request to accept (for example, if separate consumers concurrently made appointments through the system 10 for the same date and time, or if an appointment had been scheduled in-person and not yet entered into the system 10 by the provider). The system 10 may automatically send a message declining any booking request that conflicts with an accepted appointment, for example. The system 10 may also optionally monitor the accepted appointment for cancelation and send a notification to the consumer 30 who tried to schedule a conflicting appointment if the appointment becomes available. In another aspect, the system 10 may automatically and immediately confirm appointments when booked by a consumer 30. Accordingly, in an aspect, the system 10 may automatically fill a provider's schedule.

Once the provider 20 confirms the selected appointment, the system 10 may transmit a notification to the consumer 30 confirming the appointment. The system 10 may also send an appointment reminder to the consumer 30, for example, 24 hours before the scheduled appointment. This reminder may allow the consumer 30 to reconfirm the scheduled appointment. The consumer 30 may receive a notification and/or an alert, such as an e-mail message, text message, a telephone call, a message on a feed, or a message on a calendar with the appointment reminder.

When the consumer 30 reconfirms the appointment, the system 10 may notify the consumer 30 of a commitment to a no-show fee, applied in order to guarantee the appointment for the patient. For example, the system 10 may charge the consumer's credit card a full or partial amount of the service price to hold the appointment. If the patient does not arrive for the scheduled appointment, or arrives beyond an acceptable time window (e.g., thirty minutes), the system 10 may send the collected fee to the provider 20. If the patient attends the appointment, the system 10 may deduct the previously collected amount from the total amount due for the services. The system 10 may also notify the consumer 30 of the commitment to the no-show fee when initially booking the appointment. The no-show fee may be refundable until a designated time before the appointment (e.g., 24 hours), for example.

### Appointment Monitors

The system 10 may monitor the available appointments and notify a consumer 30 of any last minute or other appointments of interest that may become available. For example, the system 10 may monitor the available appointments for particular providers 20 and notify the consumer 30 when an appointment becomes available for the provider 20. The consumer 30 may also select specific time periods for the appointment, and the system 10 may notify the consumer 30 of any available appointments for the selected time periods. The consumer 30 may optionally select to participate in the appointment monitoring aspect of the system 10.

### Consumer Appointments

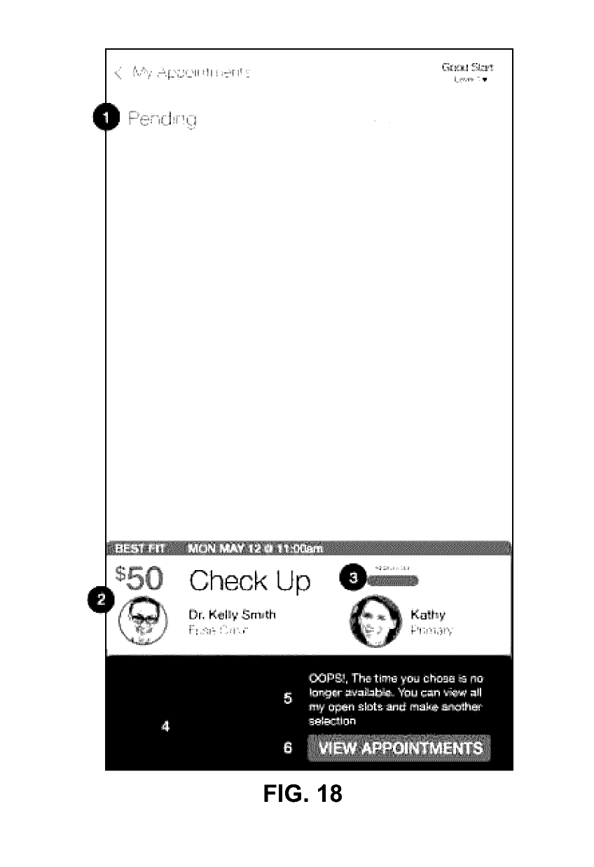

FIG. 18 illustrates an example consumer notification display. The system 10 may provide a summary of the consumer's pending, upcoming, and past appointments for the consumer 30 to view. The system 10 may also display any notifications (e.g. appointment reminders, promotions, or payment confirmations). The pending appointments may display all the requested appointments that are pending provider approval, for example. The pending appointments may display the appointment summary and indicate that the status of the appointment is “Requested.” When the appointment is confirmed by the provider 20, the status of the pending appointment may change from “Requested” to “Confirmed and Booked,” for example. In addition, the appointment may be moved from the pending appointment list to the upcoming appointment list, for example. When the appointment is not available, the system 10 may notify the consumer 30 that the appointment is not available. The system 10 may change the display of the appointment color in the pending appointments, may display a message indicating that the appointment is unavailable, and/or allow the consumer 30 to view alternative appointments by selecting a view appointments button.

The upcoming appointments may display all or a selected portion of the consumer's upcoming appointments booked through the system 10. The appointments may be displayed, for example, in chronological order with the appointment summary information displayed, but may also be ordered in a different manner by consumer selection.

The past appointments may display all or a portion of the consumer's previous appointments booked through the system 10. The appointments may be displayed in reverse chronological order with the appointment summary displayed, for example, or a differing order may be selected by the consumer 30.

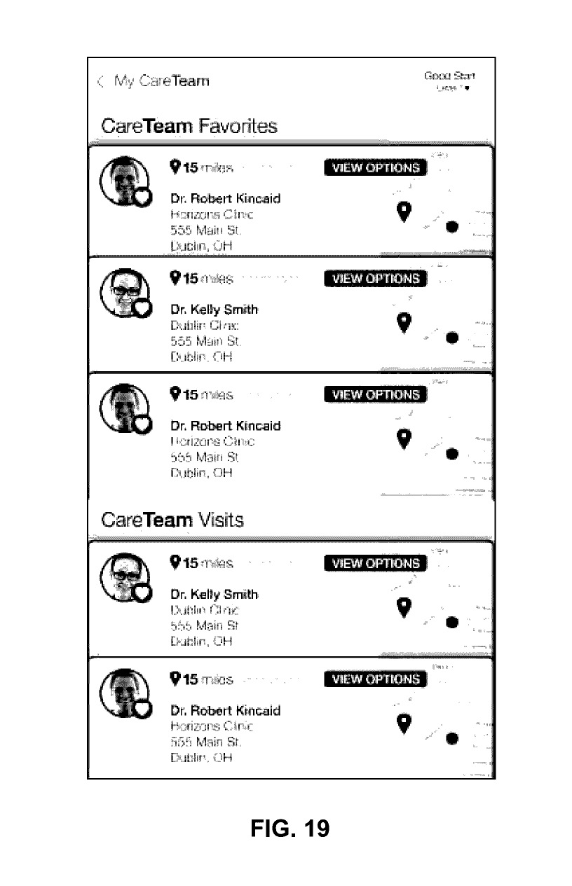

In addition, the system 10 may allow the consumer 30 to create a “Favorites” list indicating the consumer's favorite providers and/or services. As illustrated in FIG. 19, for example, the system 10 may display the consumer's selected providers 20 and visits under the favorites list, for example. The system 10 may also summarize patient spending information.

### Appointment Check-Ins

The system 10 may automatically check-in a patient for a service. The system 10 may automatically determine, for example, when a patient is within a certain distance of the provider's location by, for example, a GPS or other location component on the consumer's wireless device. In addition, for example, the consumer 30 may select a button and/or enter in the patient's location using the mobile application to notify the system 10 that the patient has arrived at the provider 20. The system 10 may send a check-in notification to the provider's system 10 so that the receptionist is notified that the patient has arrived and is checked-in for the service. The system 10 may also transmit any relevant health information the system 10 may have stored for the patient to the provider computer system 22 when the patient arrives for the service. Additionally, a provider 20 may manually check-in a patient on the provider computer system 22 when the patient arrives for their appointment.

### Payments

The system 10 may collect payments from the consumer 30 for the services provided. The system 10 may collect the credit card information for the consumer 30 and the bank details from the provider 20. For example, when the appointment is completed, the consumer 30 may be prompted to confirm the payment due to the provider 20, and the system 10 may automatically cause a transfer of the payment to the provider's bank account. The system 10 may also take out a commission from the payment to the provider 20 for facilitating the marketplace between the consumer 30 and the provider 20. In an aspect, the credit card information for a consumer 30 may be stored at a payment gateway of the payment system 80 (FIG. 1). The payment system 80 may provide the server 42 (FIG. 1) with a token that may only be used for transactions between the consumer 30 (FIG. 1) and the system 10, for example. Accordingly, the system 10 may avoid storing consumer credit card information.

Rate Providers/Consumers and/or Share Experience

Upon completion of the services or at other times thereafter, the system 10 may prompt the consumer 30 to provide a rating of the provider 20. For example, as illustrated in FIG. 19, the consumer 30 may, for example, select a number of stars or other type of rating selection and/or provide a text entry relating to rating the provider 20. The system 10 may compile the consumer ratings to determine a quality/rating parameter for the provider 20. The ratings may also be available for review by other consumers via the detailed provider 20 information. Providers 20 may be rated based on quality of the service provided, wait time, attitude/demeanor during the service, or other factors.

Likewise, upon completion of the services or at other times, the system 10 may prompt the provider 20 to provide a rating of the consumer 30. The provider 20 may, for example, select a number of stars or other type of rating selection and/or provide a text entry relating to rating the consumer 30. The system 10 may compile the provider ratings to determine a quality/rating parameter for the consumer 30. The ratings may also be available for review by other providers 20 via the detailed provider information. Consumers 30 may be rated based on punctuality, attitude/demeanor during the service, or other factors. In addition, the consumer 30 may be rated based on the retention of appointments, confirmation of appointments, prompt/delayed check-in, and/or prompt/delayed rating of the provider. For example: if a consumer 30 cancels a booked appointment more than twice in a short period of time, the system 10 may lower the consumer rating.

As discussed above, the system 10 may calculate suggested discounts or credits based on the ratings. For example, the system 10 may suggest a provider with a lower rating to offer larger discounts relative to others in the geographical area in order to attract more consumers 30. Similarly, the system 10 may offer larger discounts to consumers 30 with a higher rating in order to encourage their continued use of the system 10. Consumers 30 with higher ratings may also obtain more credits for each completed appointment. Providers 20 may decline to confirm appointments with consumers 30 below a particular rating or choose the consumer 30 with the higher rating if two or more consumers 30 have booked the same or an overlapping appointment time.

In addition, the system 10 may facilitate the sharing of the consumer's experience with others through social media accounts and/or other forums. For example, the system 10 may generate a template post indicating that the consumer 30 booked an appointment via the system 10.

### Notification Feed

The system 10 may have a notification feed that notifies the consumer 30 and/or providers 20 of actions taken by the system 10. For example, the consumer feed may receive notifications transmitted by the system 10 notifying the consumer 30 that appointments are confirmed, that the patient has been checked in to an appointment, that the patient has been checked out of an appointment, and that payment has been received for the services. In addition, the feed may notify the consumer 30 of any promotions and/or last minute appointment availabilities.

Bidding Requests from the Patient

Rather than simply selecting a priced appointment, the system 10 may provide options for receiving one or more bids from the patient and determining whether the provider 20 will accept the bid for the service. For example, the system 10 may receive a price a patient is willing to pay for a service. The system 10 may automatically calculate an acceptable price to apply to the service based on the demand for the service and the requested appointment time. For example, the acceptable price may be based on the same discounts applicable if the consumer 30 had searched for the provider's appointment. The acceptable price may also be configured with a different set of discounts applicable only to consumer bids. If the bid is within the amount the provider is willing to charge for the service, the system 10 may automatically accept the bid for the service from the patient. As such, the system 10 may optimize the revenue for the doctor by automatically scheduling patients for a provider 20 when the bid amount is within an acceptable range for the service provided.

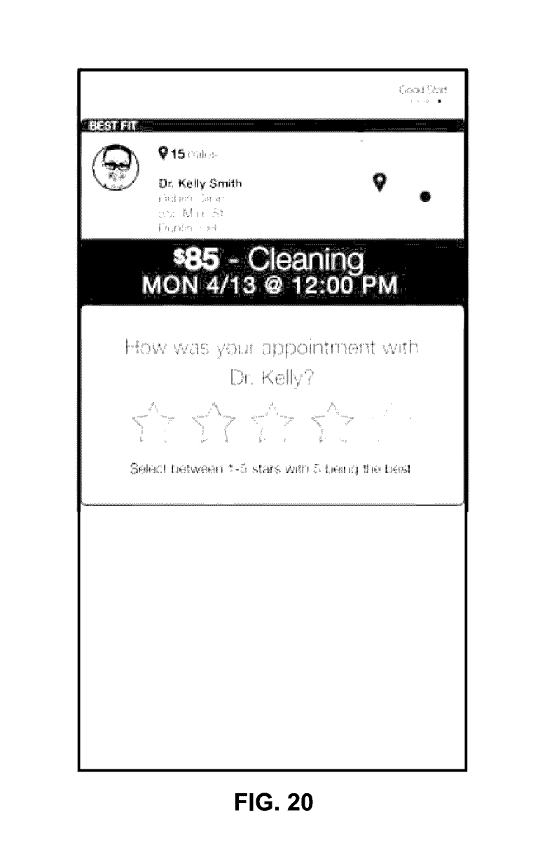

The system 10 may also allow providers 20 to manually review bids from consumers 30. FIG. 20 illustrates an example display showing bids submitted by consumers. The display may include information about the consumer 30, such as a bid amount (revenue), consumer name, patient name, consumer rating, requested service, requested times, and a time remaining to accept. A provider 20 may review the bids and select bids that are acceptable. The system 10 may send a notification to the patient when a provider 20 has accepted a bid automatically or manually.

In an aspect, the system operator may charge the patient for the service when the bid is accepted. Accordingly, a provider 20 may be willing to accept a lower bid in exchange for guaranteed revenue. The consumer 30 may be required to consent to the billing arrangement when submitting the bid.

### Computer System

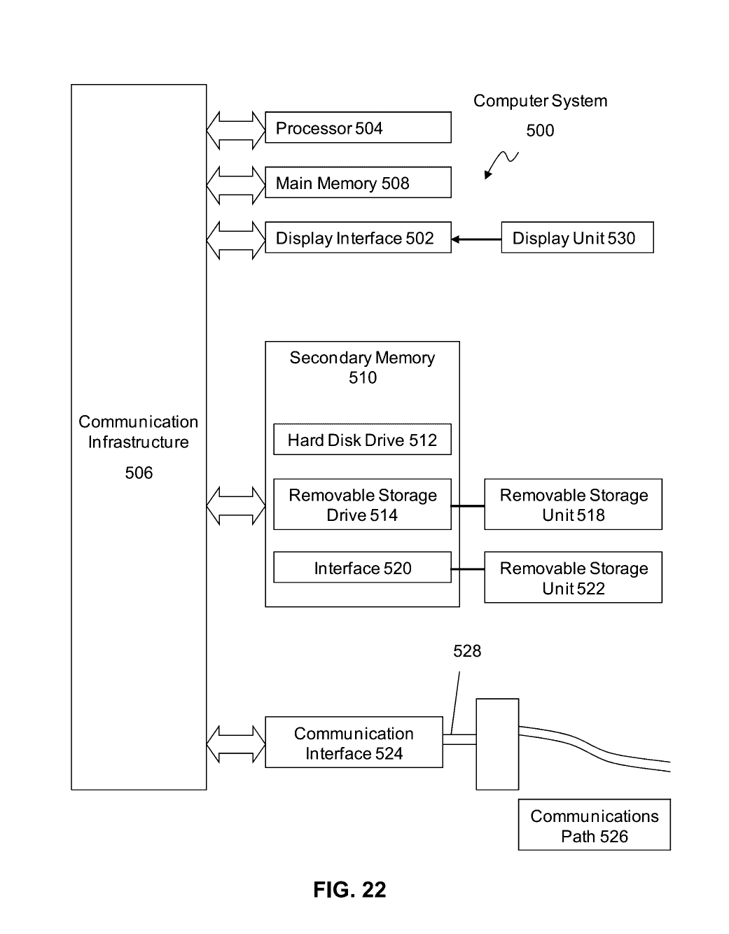

Aspects of the present invention may be implemented using hardware, software, or a combination thereof and may be implemented in one or more computer systems or other processing systems. For example, the consumer device 32, provider computer system 22, or server 42 may be implemented as a computer system or processing system. In an aspect of the present invention, features are directed toward one or more computer systems capable of carrying out the functionality described herein. An example of such a computer system 500 is shown in FIG. 22.

Computer system 500 includes one or more processors, such as processor 504. The processor 504 is coupled to a communication infrastructure 506 (e.g., a communications bus, cross-over bar, or network). Various software aspects are described in terms of this example computer system. After reading this description, it will become apparent to a person skilled in the relevant art(s) how to implement aspects hereof using other computer systems and/or architectures.

Computer system 500 may include a display interface 502 that forwards graphics, text, and other data from the communication infrastructure 506 (or from a frame buffer not shown) for display on a display unit 530. Computer system 500 may include a main memory 508, preferably random access memory (RAM), and may also include a secondary memory 510. The secondary memory 510 may include, for example, a hard disk drive 512 and/or a removable storage drive 514, e.g., an optical disk drive. The removable storage drive 514 may read from and/or write to a removable storage unit 518 in a well-known manner. As will be appreciated, the removable storage unit 518 may include a computer readable storage medium having stored therein computer software and/or data.

Alternative aspects of the present invention may include secondary memory 510 and may include other similar devices for allowing computer programs or other instructions to be loaded into computer system 500. Such devices may include, for example, a removable storage unit 522 and an interface 520. Examples of such may include a program cartridge and cartridge interface (such as that found in video game devices), a removable memory chip (such as an erasable programmable read only memory (EPROM), or programmable read only memory (PROM)) and associated socket, and other removable storage units 522 and interfaces 520, which allow software and data to be transferred from the removable storage unit 522 to computer system 500.

Computer system 500 may also include a communications interface 524. Communications interface 524 may allow software and data to be transferred among computer system 500 and external devices. Examples of communications interface 524 may include a modem, a network interface (such as an Ethernet card), a communications port, a Personal Computer Memory Card International Association (PCMCIA) slot and card, etc. Software and data transferred via communications interface 524 may be in the form of signals 528, which may be electronic, electromagnetic, optical or other signals capable of being received by communications interface 524. These signals 528 may be provided to communications interface 524 via a communications path (e.g., channel) 526. This path 526 may carry signals 528 and may be implemented using wire or cable, fiber optics, a telephone line, a cellular link, a radio frequency (RF) link and/or other communications channels. As used herein, the terms “computer program medium” and “computer usable medium” refer generally to media such as a removable storage drive 580, a hard disk installed in hard disk drive 570, and/or signals 528. These computer program products may provide software to the computer system 500. Aspects of the present invention are directed to such computer program products.

Computer programs (also referred to as computer control logic) may be stored in main memory 508 and/or secondary memory 510. Computer programs may also be received via communications interface 524. Such computer programs, when executed, may enable the computer system 500 to perform the features in accordance with aspects of the present invention, as discussed herein. In particular, the computer programs, when executed, may enable the processor 504 to perform the features in accordance with aspects of the present invention. Accordingly, such computer programs may represent controllers of the computer system 500.

Where aspects of the present invention may be implemented using software, the software may be stored in a computer program product and loaded into computer system 500 using removable storage drive 514, hard disk drive 512, or interface 520. The control logic (software), when executed by the processor 504, may cause the processor 504 to perform the functions described herein. In another aspect of the present invention, the system may be implemented primarily in hardware using, for example, hardware components, such as application specific integrated circuits (ASICs). Implementation of the hardware state machine so as to perform the functions described herein will be apparent to persons skilled in the relevant art(s). In yet another variation, aspects of the present disclosure may be implemented using a combination of both hardware and software.

While the foregoing disclosure discusses example aspects and/or features, it should be noted that various changes and modifications could be made herein without departing from the scope of the described aspects and/or features as defined by the appended claims. Furthermore, although elements of the described aspects and/or features may be described or claimed in the singular, the plural is contemplated unless limitation to the singular is explicitly stated. Additionally, all or a portion of any aspect and/or embodiment may be utilized with all or a portion of any other aspect and/or feature, unless stated otherwise.

What is claimed is:

1. An appointment scheduling system, comprising:
a consumer module that receives a selection of a service from a consumer and a geographic location of the consumer, and based on the service and location, ranks and displays available appointments for the service from a plurality of service providers within a threshold distance of the geographic location of the consumer; and
a service provider module that stores prices for services offered by the provider and a listing of available appointments belonging to a respective one of the service providers,
wherein the system ranks available appointments according to a set of weighted parameters.

2. The system of claim 1, wherein parameters comprising the set of weighted parameters used to rank the available appointment include: a price of the service, a distance of the service provider from the geographic location of the consumer, an amount of time until the available appointment, and a service provider rating.

3. The system of claim 1, wherein the service provider module discounts the price of the services based on the time of day of the available appointment, a type of the service, a percentage of available appointments in a given time period, proximity to an appointment time, a number of appointments completed by the consumer through the system, a service provider rating, or a consumer rating.

4. The system of claim 1, wherein the service provider module discounts the price of the services based on at least two of: the time of day of the available appointment, a type of the service, a percentage of available appointments in a given time period, proximity to an appointment time, a number of appointments completed by the consumer through the system, a service provider rating, and a consumer rating.

5. The system of claim 1, wherein the service provider module discounts the price of the services after determining the consumer completed a requisite number of appointments through the system while attaining a predetermined minimum average consumer rating.

6. The system of claim 1, wherein the consumer module hides display of at least one of the available appointments from the consumer based on a consumer rating of the consumer, and wherein the consumer rating is based on at least one of: a number of appointments completed through the system, a rating given to the consumer by the services providers during previous appointments, a number of times the consumer arrived late for appointments scheduled through the system, and a number of times the consumer did not arrive for appointments scheduled through the system.

7. The system of claim 4, wherein the consumer rating is based on at least one of: a number of appointments completed by the consumer through the system, a rating given to the consumer by the services providers during previous appointments, a number of times the consumer arrived late for appointments scheduled through the system, and a number of times the consumer did not arrive for appointments scheduled through the system.

8. The system of claim 1, wherein the consumer module classifies the ranked display of the available appointments by groups, and wherein each of the groups is displayed with at least one of a different color, brightness or size.

9. The system of claim 2, wherein the system calculates initial weights of the parameters relative to each other based on input by the consumer of importance of each of the parameters individually.

10. The system of claim 9, wherein after the consumer has completed at least a first appointment through the system, the system automatically varies the initial weights of the parameters based on at least one of a selection history of the consumer, a selection history of other consumers within the threshold distance, or a selection history of other consumers with similar personal characteristics as the consumer.

11. The system of claim 2, wherein the system automatically varies relative weightings of the parameters based on at least one of a selection history of the consumer, a selection history of other consumers within the threshold distance, or a selection history of other consumers with similar personal characteristics as the consumer.

12. The system of claim 1, wherein the set of weighted parameters used to rank the available appointment consists of: a price of the service, a distance of the service provider from the geographic location of the consumer, an amount of time until the available appointment, and a service provider rating.

13. The system of claim 1, wherein the service provider module automatically checks-in the consumer for an appointment booked through the system by determining that the consumer is within a predetermined distance of a geographic location of the service provider within a predetermined range of a booked appointment time.

14. A method of scheduling an appointment, comprising:
receiving a selection of a service from a consumer using a computing device;
receiving a geographic location of the consumer;
determining available appointments for the service from a plurality of service providers within a threshold distance of the geographic location of the consumer;
ranking the available appointments according to a set of weighted parameters;
displaying the available appointments to the consumer in order of the ranking;
receiving a selection of an available appointment;
confirming the selected available appointment with a provider of the available appointment; and
notifying the consumer that the appointment has been confirmed.

15. The method of claim 14, wherein the set of weighted parameters used to rank the available appointment includes a price of the service, a distance of the service provider from the geographic location of the consumer, and a service provider rating.

16. The method of claim 15, wherein the distance of the service provider from the geographic location of the consumer is calculated in real-time by the computing device.
